(19) United States
(12) Patent Application Publication
(10) Pub. No.: US 2013/0252334 A1
(43) Pub. Date: Sep. 26, 2013

(54) METHOD OF USING TET-INDUCIBLE TRANSGENES

(71) Applicant: Marc Halterman, Penfield, NY (US)

(72) Inventor: Marc Halterman, Penfield, NY (US)

(73) Assignee: UNIVERSITY OF ROCHESTER, Rochester, NY (US)

(21) Appl. No.: 13/877,476

(22) PCT Filed: Oct. 10, 2011
(86) PCT No.: PCT/US11/55583

§ 371 Date: Jun. 14, 2013

Related U.S. Application Data
(60) Provisional application No. 61/391,457, filed on Oct. 8, 2010.

(51) Int. Cl. C12N 15/63

(52) U.S. Cl. CPC C12N 15/635
USPC 435/375; 435/320.1

(57) ABSTRACT

The invention relates to a method of using an inducible transgene in a cell, where the inducible transgene expresses a gene product having activity on a cell death or cell survival pathway. The method includes exposing the cell to an agent that is capable of inducing the inducible transgene but also has direct activity on a cell death or cell survival pathway, said exposing being carried out in a manner effective to induce expression of the transgene while substantially minimizing said direct activity of the agent. Host cells and kits for carrying out the method are also disclosed.

This application claims the benefit of U.S. Provisional Patent Application Ser. No. 61/391,457, filed Oct. 8, 2010, which is hereby incorporated by reference in its entirety.

This invention was made with government support under National Institutes of Health/National Institute of Neurological Disorders and Stroke under R00NS060764. The government has certain rights in the invention.

### FIELD OF THE INVENTION

The present invention relates to an improved method for the study of apoptosis-related genes.

### BACKGROUND OF THE INVENTION

The use of ligand-regulated expression systems to study the function of unknown genes has become commonplace. With the option to either induce or repress gene expression, such methods provide the means to control both the timing and level of gene expression both in culture and in vivo. One of the first regulated expression systems reported is based on the tetracycline resistance operon tet (tetO) contained within the Escherichia coli transposon Tn10. In this system, the Ptet promoter, in addition to driving expression of the TetA protein that pumps tetracycline out of the cell, also regulates expression of the Tet repressor (TetR) (Furth et al., “Temporal Control of Gene Expression in Transgenic Mice by a Tetracycline-Responsive Promoter,” Proc. Nat'l Acad. Sci. 91(20):9302-9306 (1994); Gossen et al., “Tight Control of Gene Expression in Mammalian Cells by Tetracycline-Responsive Promoters,” Proc. Nat'l Acad. Sci. 89(12):5547-5551 (1992); and Gossen et al., “Anhydrotetracycline, a Novel Effector for Tetracycline Controlled Gene Expression Systems in Eukaryotic Cells,” Nucleic Acids Res. 21:4411-4412 (1993). In the presence of ligand, TetR is unable to bind to tet operator sequences (tetO), thus allowing transcription to proceed. Fusion of the TetR repressor with the transactivation domain from VP16 converts TetR into an activator protein (tTA) that retains affinity for tetO sequences in the absence of ligand. In this system, the Pₜₑₜ promoter regulates expression of the TetR repressor and the TetA protein that pumps tetracycline out of the cell. Fusion of the TetR repressor with the transactivation domain from viral protein VP16 creates a transcriptional activator protein that is both drug responsive, and able to bind to tetracycline response promoter elements (tetO) (Paillard, “‘Tet-on’: A Gene Switch for the Exogenous Regulation of Transgene Expression,” Hum. Gene Ther. 9(7):983-985 (1998) and Molin et al., “Two Novel Adenovirus Vector Systems Permitting Regulated Protein Expression in Gene Transfer Experiments,” J. Virol. 72(10):8358-8361 (1998)). Conversely, in the case of the Tet-Off system, fusion of the rTA transactivator with elements harboring strong repressor activity shuts down constitutive gene expression in the presence of ligand. Although tetracycline is the prototypical ligand, related analogs gate tet-inducible systems as well.

Subsequently, Gossen (Gossen et al., “Transcriptional Activation by Tetracyclines in Mammalian Cells,” Science 268:1766-1769 (1995)) and other authors (Jantzie et al., “Doxycycline Reduces Cleaved Caspase-3 and Microglial Activation in an Animal Model of Neonatal Hypoxia-Ischemia,” J. Cereb. Blood Flow Metab. 25:314-324 (2005); Jin et al., “Vascular Endothelial Growth Factor: Direct Neuroprotective Effect in in vitro Ischemia,” Proc. Natl. Acad. Sci. U.S.A. 97:10242-10247 (2000); Keilhoff et al., “Minocycline Protects Schwann Cells from Ischemia-Like Injury and Promotes Axonal Outgrowth in Bioartificial Nerve Grafts Lacking Wallerian Degeneration,” Exp. Neurol. 212:189-200 (2008); Lamartina et al., “Stringent Control of Gene Expression in vivo by Using Novel Doxycycline-Dependent Trans-Activators,” Hum. Gene Ther. 13:199-210 (2002); Lee et al., “Neuronal Properties and Trophic Activities of Immortalized Hippocampal Cells from Embryonic and Young Adult Mice,” J. Neurosci. 10:1779-1787 (1990); Lee et al., “Immortalized Young Adult Neurons from the Septal Region: Generation and Characterization,” Brain Res. Dev. Brain Res. 52:219-228 (1990); Marchetti et al., “Inducible Expression of a MAP Kinase Phosphatase-3-GFP Chimera Specifically Blunts Fibroblast Growth and ras-Dependent Tumor Formation in Nude Mice,” J. Cell Physiol. 199:441-450 (2004); Nelson et al., “Versatile and Facile Synthesis of Diverse Semisynthetic Tetracycline Derivatives via Pd-Catalyzed Reactions,” J. Org. Chem. 68:5838-5851 (2003); and Paillard, “‘Tet-On’: A Gene Switch for the Exogenous Regulation of Transgene Expression,” Hum. Gene Ther. 9:983-985 (1998)) have developed transactivators with reversed DNA binding properties (rtTA), creating a dox-inducible platform. Additional mutations that improve the inducibility and reduce basal transgene expression have since been described (Urlinger et al., “Exploring the Sequence Space for Tetracycline-Dependent Transcriptional Activators: Novel Mutations Yield Expanded Range and Sensitivity,” Proc. Natl. Acad. Sci. U.S.A. 97:7963-7968 (2000)). Importantly, although tetracycline is the prototypical ligand, related analogs also gate tet-inducible systems as well. For example, Zhu (Zhu et al, “Silencing and Un-Silencing of Tetracycline-Controlled Genes in Neurons,” PLoS One 2:e533 (2007)) reported taking advantage of the hydrophobic properties of the novel tetracycline analog 9-t-butyl doxycycline (9-TB) to regulate gene expression within the central nervous system of tet-reporter transgenic mice.

Although their use is contraindicated in childhood and pregnancy, tetracycline compounds have been used to treat a variety of medical conditions and may ultimately be suitable for use in clinical protocols involving regulated expression of therapeutic genes expressed from viral and cell-based vectors. Interestingly, in addition to their broad-spectrum antibacterial and antiprotozoal effects, tetracyclines exert both neuroprotective and anti-inflammatory effects on somatic tissues (Keilhoff et al., “Minocycline Protects Schwann Cells from Ischemia-Like Injury and Promotes Axonal Outgrowth in Bioartificial Nerve Grafts Lacking Wallerian Degeneration,” Exp. Neurol. 212: 189-200 (2008)). For example, both doxycycline and minocyline have been linked with inhibition of matrix metalloproteases and pro-caspase-3 activation (Jantzie et al., “Doxycycline Reduces Cleaved Caspase-3 and Microglial Activation in an Animal Model of Neonatal Hypoxia-Ischemia,” J. Cereb. Blood Flow Metab. 25:314-324 (2005)). These observations have led to speculations that these compounds may benefit patients with noninfectious, chronic degenerative disorders, including multiple sclerosis, amyotrophic lateral sclerosis (ALS), and stroke (Furth et al., “Temporal Control of Gene Expression in Transgenic Mice by a Tetracycline-Responsive Promoter,” Proc. Natl. Acad. Sci. U.S. A 91:9302-9306 (1994); Gossen et al., “Anhydrotetracycline, a Novel Effector for Tetracycline Controlled Gene Expression Systems in Eukaryotic Cells,” Nucleic Acids Res. 21:4411-4412 (1993); Gossen et al., “Tight Control of Gene Expression in Mammalian Cells by Tetracycline-Responsive Promoters,” Proc. Natl. Acad. Sci. U.S.A. 89:5547-5551 (1992); Gossen et al., “Transcriptional Activation by Tetracyclines in Mammalian Cells,” Science 268:1766-1769 (1995); Jantzie et al., “Doxycycline Reduces Cleaved Caspase-3 and Microglial Activation in an Animal Model of Neonatal Hypoxia-Ischemia,” J. Cereb. Blood Flow Metab. 25:314-324 (2005); Jin et al. “Vascular Endothelial Growth Factor: Direct Neuroprotective Effect in in vitro Ischemia,” Proc. Natl. Acad. Sci. U.S.A. 97:10242-10247 (2000); Keilhoff et al., “Minocycline Protects Schwann Cells from Ischemia-Like Injury and Promotes Axonal Outgrowth in Bioartificial Nerve Grafts Lacking Wallerian Degeneration,” Exp. Neurol. 212:189-200 (2008); Lamartina et al., “Stringent Control of Gene Expression in vivo by Using Novel Doxycycline-Dependent Trans-Activators,” Hum. Gene Ther. 13:199-210 (2002); Lee et al., “Neuronal Properties and Trophic Activities of immortalized Hippocampal Cells from Embryonic and Young Adult Mice,” J. Neurosci. 10:1779-1787 (1990); Lee et al., “Immortalized Young Adult Neurons from the Septal Region: Generation and Characterization,” Brain Res. Dev. Brain Res. 52:219-228 (1990); Marchetti et al., “Inducible Expression of a MAP Kinase Phosphatase-3-GFP Chimera Specifically Blunts Fibroblast Growth and ras-Dependent Tumor Formation in Nude Mice,” J. Cell Physiol. 199:441-450 (2004); Nelson et al., “Versatile and Facile Synthesis of Diverse Semisynthetic Tetracycline Derivatives via Pd-Catalyzed,” Reactions. J. Org. Chem. 68:5838-5851 (2003); Paillard, “‘Tet-On’: A Gene Switch for the Exogenous Regulation of Transgene Expression,” Hum. Gene Ther. 9:983-985 (1998); Schmidt-Kastner et al., “Neuroglobin mRNA Expression after Transient Global Brain Ischemia and Prolonged Hypoxia in Cell Culture,” Brain Res. 1103:173-180 (2006); Shoshani et al., “Identification of a Novel Hypoxia-inducible Factor 1-Responsive Gene, RTP801, Involved in Apoptosis,” Mol. Cell. Biol. 22:2283-2293 (2002); Strathdee et al., “Efficient Control of Tetracycline-Responsive Gene Expression from an Autoregulated Bi-Directional Expression Vector,” Gene 229:21-29 (1999); Tikka et al., “Minocycline Provides Neuroprotection against N-methyl-D-Aspartate Neurotoxicity by inhibiting Microglia,” J. Immunol. 166:7527-7533 (2001); and Domercq et al., “Neuroprotection by Tetracyclines,” Trends Pharmacol. Sci. 25:609-612 (2004)).

One of the principal advantages of an inducible gene expression system is having the ability to generate stable transfectants harboring toxic genes where conventional constitutive gene expression systems might otherwise fail (Marchetti et al., “Inducible Expression of a MAP Kinase Phosphatase-3-GFP Chimera Specifically Blunts Fibroblast Growth and ras-Dependent Tumor Formation in Nude Mice,” J. Cell Physiol. 199:441-450 (2004); Nelson et al., “Versatile and Facile Synthesis of Diverse Semisynthetic Tetracycline Derivatives via Pd-Catalyzed,” Reactions. J. Org. Chem. 68:5838-5851 (2003); Paillard, “‘Tet-On’: A Gene Switch for the Exogenous Regulation of Transgene Expression,” Hum. Gene Ther. 9:983-985 (1998); and Schmidt-Kastner et al., “Neuroglobin mRNA Expression after Transient Global Brain ischemia and Prolonged Hypoxia in Cell Culture,” Brain Res. 1103:173-180 (2006)). In studying the role of hypoxia-induced gene expression on cell survival, it was discovered that dox exposure itself inhibited stress-induced caspase-3 cleavage in control cell lines, confounding the ability to isolate effects of the gene of interest.

Given their tight regulatory control, it would be desirable to identify a way to use these inducible systems in a manner that at least minimizes or, more preferably, eliminates the direct activity of the inducing agent on cell death or cell survival pathways. The present invention is directed to overcoming these and other deficiencies in the art.

### SUMMARY OF THE INVENTION

A first aspect of the present invention involves a method of using an inducible transgene in a cell. The method includes the steps of providing a cell comprising an inducible transgene that expresses a gene product having activity on a cell death or cell survival pathway; and exposing the cell to an agent that is capable of inducing the inducible transgene but also has direct activity on a cell death or cell survival pathway, said exposing being carried out in a manner effective to induce expression of the transgene while substantially minimizing said direct activity of the agent.

A second aspect of the present invention relates to a host cell that includes an inducible transgene comprising a promoter-effective nucleic acid molecule that includes a tetracycline-responsive element (TRE), the promoter-effective nucleic acid molecule being operatively coupled to a nucleic acid molecule that encodes a gene product having activity on a cell death or cell survival pathway.

A third aspect of the present invention relates to a kit comprising a response plasmid comprising (i) a promoter-effective nucleic acid molecule that includes a tetracycline-responsive element (TRE) and (ii) an insertion site suitable for introduction of an open reading frame of interest. The kit includes a regulator plasmid comprising a transgene that encodes a fusion protein comprising a first polypeptide that binds to the TRE in the presence or absence of a substituted tetracycline compound and is operatively linked to a second polypeptide that regulates transcription in cells. The kit further includes instructions for preparing a transgenic cell that can express the open reading frame of interest and the fusion protein, and exposing the transgenic cell to a substituted tetracycline compound according to a schedule that is effective to induce expression of the open reading frame of interest while substantially minimizing direct activity of the substituted tetracycline compound on a cell death or cell survival pathway.

Inducible gene expression systems are particularly useful for the functional characterization of genes with putative toxic properties, particularly those capable of modifying a cell death or cell survival pathway. In the course of studying the role of hypoxia-regulated gene expression on cell survival using the tetracycline inducible (Tet-On) system, it was noted that exposure to tetracycline derivative compounds inhibited caspase-3 cleavage directly in control stable reporter lines. To limit this confounding off-target effect, an in vitro pulse-dose was devised that delayed injury protocol using the tetracycline analog 9-t-butyl doxycycline (9-TB). While 9-TB induced higher transgene levels compared to matched concentrations of doxycycline, continuous exposure to both drugs inhibited caspase-3 cleavage in hypoxic samples. However, results from pulse-dose analyses indicate that brief exposure to 9-TB (up to about 12 hours) paired with a prolonged washout period (exceeding about 24 hours) resulted in robust transgene expression while substantially minimizing the impact continuous ligand exposure has on levels of caspase-3 cleavage. This protocol will allow for enhancement of studies based on the tet-regulated gene expression system, especially in situations where cell death (or cell survival) is used as a primary endpoint.

### BRIEF DESCRIPTION OF THE DRAWINGS

FIGS. 1A-C are comparisons of doxycycline and 9-t-butyl doxycycline (9TB) on regulated GFP expression from a tet-ON reporter cell line. FIG. 1A is a schematic of the exposure paradigm used to compare ligand effects on stable reporter gene expression. Plating, drug treatment (Rx), and analysis time points are shown.

FIG. 1B shows that drug exposure does not adversely affect cultures under standard conditions. Normoxic tet-ON GFP stable cells were cultured in the presence of 1 μg/ml of either Dox or 9-TB for a total of 40 hours and evaluated by bright phase microscopy. FIG. 1C is a Western analysis of lysates harvested from dox- and 9-TB-treated, tet-ON GFP cultures demonstrating relative ligand-dependent dose responses. Actin blotting confirms equivalent sample loading.

FIG. 2 shows a Western blot illustrating the effects of tetracycline compounds on hypoxia-induced caspase-3 cleavage. The tet-ON inducible stable cell line was exposed to varying concentrations of ligand (0.5 vs 2.5 μg/mL) and parallel cultures were exposed to either normoxic or hypoxic (0.5% O₂) conditions for 18 hours. Lysates were analyzed by Western blotting for both transgene induction (GFP) as well as levels of cleaved caspase-3 (cCasp3).

FIGS. 3A-B illustrate one embodiment of pulse dosing with 9-TB or doxycycline, which induces transgene expression and mitigates the inhibitory effects of ligand on hypoxia-induced caspase-3 cleavage. FIG. 3A is a schematic diagram of the pulse-dosing strategy. Cells were plated and exposed to either doxycycline or 9-TB (2.5 μg/mL; Rx) for 12 hours prior to media replacement (i.e., washout 16 hours) prior to hypoxic exposure (0.5% O₂, 24 hours). FIG. 3B is a Western blot for dox- and 9-TB-regulated GFP expression and ligand-mediated inhibition of hypoxia-induced caspase-3 cleavage.

FIGS. 4A-B illustrate several embodiments of pulse dosing and the influence of the washout period length on GFP expression and caspase-3 cleavage profiles. FIG. 4A is a protocol timeline. Cultures were drug exposed (9-TB; 2.5 μg/mL; 12 hours) at the time of plating and incubated for 12 hours prior to full media exchange. Following a washout period of variable length (0, 12 and 24 hours) cultures were continued in either normoxic conditions (open bars) or exposed to hypoxia (black bars; 0.5% O₂, 18 hours) prior to analysis. The time line in terms of days in culture is also shown in FIG. 4A. In FIG. 4B, a Western blot demonstrates the profile of transgene induction and caspase-3 cleavage.

FIGS. 5A-C illustrate several embodiments of pulse dosing and compare the influence of the drug exposure and washout period lengths (i.e., optimizing the duration of 9-TB pulse) to maximize transgene expression. FIG. 5A is a protocol timeline. Two doses of ligand (0.25 and 2.5 ug/ml) were compared using two pulse-dosing periods (6 hours vs. 12 hours) prior to a washout period for a total of 48 hours under normoxic conditions. In FIG. 5B, Western analysis (n=2) of duplicate samples for GFP expression levels relative to actin are shown. In FIG. 5C, effects of low dose 9-TB (2.5 μg/mL) on caspase processing relative to untreated controls (n=2) is shown. The fold induction of caspase-3 levels in hypoxic samples relative to normoxic controls is shown.

### DETAILED DESCRIPTION OF THE INVENTION

The present invention relates to methods and products useful for studying the role of transgene expression products on a cell death or cell survival pathway.

One aspect of the present invention involves a method of using an inducible transgene in a cell. The method includes providing a cell that contains an inducible transgene that is capable of expressing a gene product having activity on a cell death or cell survival pathway, and exposing the cell to an agent that is capable of inducing the inducible transgene but also has direct activity on a cell death or cell survival pathway, where the exposing is carried out in a manner effective to induce expression of the transgene while substantially minimizing the direct activity of the agent.

As used herein, the phrase “substantially minimizing” means that the direct activity of the agent is less than about 50% of its full activity (on the cell death or cell survival pathway), more preferably less than about 40%, 30%, 20%, or even less that about 10% of its full activity, when the cell is exposed to the agent continuously over a period of time in excess of 6 hours. Most preferably, the direct activity of the agent is less than about 5% of its full activity (on the cell death or cell survival pathway) when the cell is exposed to the agent continuously over a period of time in excess of about 6 hours. Measurement of the agent's direct activity can be performed using any assay suitable for measuring a byproduct of the agent's direct activity or the compounds or reagents that are acted upon by the agent. Examples of suitable assays include, without limitation, immunoassays, analysis of gene expression by quantitative PCR or whole genome sequencing, as well as analyses intended to quantify cell survival or injury in cytotoxicity models.

The inducible transgene includes a coding region (or open reading frame) whose expression is regulated by a regulatory sequence that includes a tetracycline-responsive element (TRE). The recombinant cell that contains the inducible transgene further includes a fusion protein or non-covalently associated proteins that include a first polypeptide that binds to the TRE in the presence or absence of a substituted tetracycline compound and a second polypeptide that regulates transcription in cells.

The term “regulatory sequence” is art-recognized and is intended to include promoters, enhancers and other expression control elements (e.g., polyadenylation signals). Such regulatory sequences are known to those skilled in the art and are described in Goeddel, Gene Expression Technology: Methods in Enzymology 185, Academic Press, San Diego, Calif. (1990), which is hereby incorporated by reference in its entirety. Regulatory sequences include those which direct constitutive expression of a nucleotide sequence in many types of host cells and those which direct expression of the nucleotide sequence only in certain host cells (e.g., tissue-specific regulatory sequences). Other elements included in the design of a particular expression vector can depend on such factors as the choice of the host cell to be transformed, the level of expression of protein desired, etc. The expression constructs of the invention can be introduced into host cells to thereby produce proteins or peptides, including fusion proteins or peptides, and encoded nucleic acids, as described herein.

The regulatory sequence may include a minimal promoter sequence of which only a downstream part is transcribed and which serves, at least in part, to position the transcriptional machinery for transcription. The minimal promoter sequence is linked to the transcribed sequence of interest in a 5′ to 3′ direction by phosphodiester bonds (i.e., the promoter is located upstream of the transcribed sequence of interest) to form a contiguous nucleotide sequence. The term “minimal promoter” includes partial promoter sequences that define the start site of transcription for the linked sequence to be transcribed but which by itself is not capable of initiating transcription efficiently, if at all. Thus, the activity of such a minimal promoter is dependent upon the binding of a transcriptional activator (such as the tetracycline-inducible fusion protein of the invention) to an operatively linked regulatory sequence (such as one or more tet operator sequences). In one embodiment, the minimal promoter is from the human cytomegalovirus as described in Boshart et al., Cell 41:521-530 (1985), which is hereby incorporated by reference in its entirety. Preferably, nucleotide positions between about +75 to −53 and +75 to −31 of human CMV are used. Other suitable minimal promoters are known in the art or can be identified by standard techniques. For example, a functional promoter which activates transcription of a contiguously linked reporter gene (e.g., chloramphenicol acetyl transferase, β-galactosidase or luciferase) can be progressively deleted until it no longer activates expression of the reporter gene, alone, but rather requires the presence of an additional regulatory sequence(s).

In the tetracycline regulated gene expression system, transcription of a gene is modulated by a transcriptional regulator, e.g., activated by an activator protein (or reverse transactivator protein) or inhibited by transcriptional silencer proteins. The transactivators and silencers are fusion proteins or non-covalently associated proteins. Certain methods of the invention thus feature fusion proteins and nucleic acids (e.g., DNA) encoding fusion proteins or non-covalently associated proteins. The term “fusion protein” includes a polypeptide comprising an amino acid sequence derived from two different polypeptides, typically from different sources (e.g., different cells and/or different organisms) which are operatively linked together. For such polypeptides, the term “operatively linked” is intended to mean that the two polypeptides are connected in a manner such that each polypeptide can serve its intended function. Typically, the two polypeptides are covalently attached through peptide bonds. The fusion protein is generally produced by standard recombinant DNA techniques. For example, a DNA molecule encoding the first polypeptide is ligated to another DNA molecule encoding the second polypeptide, and the resultant hybrid DNA molecule is expressed in a host cell to produce the fusion protein. The DNA molecules are ligated to each other in a 5′ to 3′ orientation such that, after ligation, the translational frame of the encoded polypeptides is not altered (i.e., the DNA molecules are ligated to each other in-frame).

In one embodiment, transcription of a gene or gene product is activated by a tetracycline controlled transcriptional activator protein (tTA) or a reverse tetracycline controlled transcriptional activator protein (rtTA), both also referred to herein simply as transactivators. In the absence of a tetracycline a tTA binds to a TRE and activates expression from the target nucleic acid sequence. Conversely, the rtTA only recognizes the TRE in the presence of a tetracycline and, accordingly, transcription of the target nucleic acid sequence is stimulated by the rtTA only in the presence of a tetracycline.

The methods of the invention may also feature transcriptional silencer fusion proteins. The inhibitor fusion proteins of the methods of the invention are constructed similarly to the transcriptional regulator fusion proteins of the invention but instead of containing a polypeptide domain that stimulates transcription in a cell, the inhibitor fusion proteins contain a polypeptide domain that inhibits transcription in eukaryotic cells. The inhibitor fusion proteins are used to downregulate the expression of a transgene operably linked to tetO sequences. For example, when a tetO-linked gene is introduced into a host cell or animal, the level of basal, constitutive expression of the gene may vary depending upon the type of cell or tissue in which the gene is introduced and on the site of integration of the gene. Alternatively, constitutive expression of endogenous genes into which tetO sequences have been introduced may vary depending upon the strength of additional endogenous regulatory sequences in the vicinity. The inhibitor fusion proteins described herein provide compositions that can be used to inhibit the expression of such tetO-linked genes in a controlled manner.

For example, the inhibitor fusion protein of the methods of the invention may comprise a first polypeptide that binds to tet operator sequences in the absence, but not the presence, of a substituted tetracycline compound operatively linked to a heterologous second polypeptide that inhibits transcription in eukaryotic cells. Alternatively, the inhibitor fusion protein may comprise a first polypeptide that binds to tet operator sequences in the presence, but not the absence, of a substituted tetracycline compound operatively linked to a heterologous second polypeptide that inhibits transcription in eukaryotic cells.

In one embodiment, a transactivator fusion protein featured in certain methods of the invention is composed, in part, of a first polypeptide which binds to a Tet operator sequence in the absence of a substituted tetracycline compound of the invention.

In one embodiment, e.g., when making a tTA fusion protein, the first polypeptide is a wild-type Tet repressor (which binds to tet operator sequences in the absence but not the presence of tetracycline). A wild-type Tet repressor of any class (e.g., A, B, C, D or E) may be used as the first polypeptide. In light of the high degree of sequence conservation (at least 80%) among members of each class of Tet repressor, a single member of each class of Tet repressor is used herein as representative of the entire class. Accordingly, the teaching of the present invention with respect to a specific member of a Tet repressor class is directly applicable to all members of that class. As used herein, the TetR(A) class is represented by the Tet repressor carried on the Tn1721 transposon (Allmeir et al., “Complete Nucleotide Sequence of Tn1721: Gene Organization and a Novel Gene Product with Features of a Chemotaxis Protein,” Gene 111(1):11-20 (1992); Genbank accession number X61367, each of which is hereby incorporated by reference in its entirety); the TetR(B) class is represented by a Tet repressor encoded by a Tn10 tetracycline resistance determinant (Postle et al., “Nucleotide Sequence of the Repressor Gene of the TN10 Tetracycline Resistance Determinant,” Nucleic Acids Research 12(12): 4849-63 (1984); Genbank Accession No. X00694, each of which is hereby incorporated by reference in its entirety); the TetR(C) class is represented by the tetracycline repressor of the plasmid pSC 101 (Brow et al., “The tetracycline repressor of pSC101,” Mol. Biol. Evol. 2(1):1-12 (1985); Genbank Accession No. M36272, each of which is hereby incorporated by reference in its entirety); the TetR(D) class is represented by the Tet repressor identified in Salmonella ordonez (Allard et al., “Nucleotide Sequence of Class D Tetracycline Resistance Genes From Salmonella ordonez,” Mol. Gen. Genet. 237(1-2):301-5 (1993); Genbank Accession No. X65876, each of which is hereby incorporated by reference in its entirety); the TetR(E) class is represented by a Tet repressor isolated from a member of Enterobacteriaceae (Tovar et al., “Identification and Nucleotide Sequence of the Class E Tet Regulatory Elements and Operator and Inducer Binding of the Encoded Purified Tet Repressor,” Mol. Gen. Genet. 215(1):76-80 (1988); Genbank Accession No. M34933, each of which is hereby incorporated by reference in its entirety); the TetR(G) class is represented by a Tet repressor identified in Vibrio anguillarum (Zhao et al., “Nucleotide Sequence Analysis of the Class G Tetracycline Resistance Determinant From Vibrio anguillarum,” Microbiol. Immunol. 36(10):1051-60 (1992); Genbank Accession No. S52438, each of which is hereby incorporated by reference in its entirety); the TetR(H) class is represented by a Tet repressor encoded by plasmid pMV111 isolated from Pasteurella multocida (Hansen et al., “A New Tetracycline Resistance Determinant, Tet H, From Pasteurella multocida Specifying Active Efflux of Tetracycline,” Antimicrob. Agents. Chemother. 37(12):2699-705 (1993); Genbank Accession No. U00792, each of which is hereby incorporated by reference in its entirety); the TetR(J) class is represented by a Tet repressor cloned from Proteus mirabilis (Magalhaes et al., “A New Tetracycline Resistance Determinant Cloned From Proteus mirabilis,” Biochim. Biophys. Acta 1443(1-2):262-66 (1998); Genbank Accession No. AF038993, each of which is hereby incorporated by reference in its entirety); and the TetR(Z) class is represented by a Tet repressor encoded by the pAG1 plasmid isolated from the gram-positive organism Corynebacterium glutamicum (Tauch et al., “TetZ, A New Tetracycline Resistance Determinant Discovered in Gram-Positive Bacteria, Shows High Homology to Gram-Negative Regulated Efflux Systems,” Plasmid 44(3):285-91 (2000); Genbank Accession No. AAD25064, each of which is hereby incorporated by reference in its entirety).

In another embodiment, a transactivator fusion protein featured in certain methods of the invention is composed, in part, of a first polypeptide which binds to a tet operator sequence in the presence of a substituted tetracycline compound of the invention. Accordingly, in one embodiment, e.g., when making an rtTA fusion protein, the first polypeptide of the fusion protein is a mutated Tet repressor. The amino acid difference(s) between a mutated Tet repressor and a wild-type Tet repressor may be substitution of one or more amino acids, deletion of one or more amino acids or addition of one or more amino acids. Preferably, the mutated Tet repressor of the invention has the following functional properties: 1) the polypeptide can bind to a tet operator sequence, i.e., it retains the DNA binding specificity of a wild-type Tet repressor; and 2) it is regulated in a reverse manner by substituted tetracycline compounds compared to a wild-type Tet repressor, i.e., the mutated Tet repressor binds to a tet operator sequence only the presence of a substituted tetracycline compound rather than in the absence of a substituted tetracycline compound.

In an embodiment, a mutated Tet repressor having the functional properties described above is created by substitution of amino acid residues in the sequence of a wild-type Tet repressor. For example, as described in U.S. Pat. No. 5,789,156 to Bujard et al., a Tn10-derived Tet repressor having amino acid substitutions at amino acid positions 71, 95, 101 and 102 has the desired functional properties and, thus, can be used as the first polypeptide in the transcriptional regulator fusion protein of the invention. Mutation of fewer than all four of these amino acid positions may be sufficient to achieve a Tet repressor with the desired functional properties. Accordingly, in the embodiment, a Tet repressor is preferably mutated at least one of these positions. Other amino acid substitutions, deletions or additions at these or other amino acid positions which retain the desired functional properties of the mutated Tet repressor are within the scope of the methods of the invention. The crystal structure of a Tet repressor-tetracycline complex, as described in Hinrichs et al., “Structure of the Tet Repressor-Tetracycline Complex and Regulation of Antibiotic Resistance,” Science 264:418-420 (1994), which is hereby incorporated by reference in its entirety, can be used for rational design of mutated Tet repressors. Based upon this structure, amino acid position 71 is located outside the tetracycline binding pocket, suggesting mutation at this site may not be necessary to achieve the desired functional properties of a mutated Tet repressor of the invention. In contrast, amino acid positions 95, 101 and 102 are located within the conserved tetracycline binding pocket. Thus, the tetracycline binding pocket of a Tet repressor may be targeted for mutation to create a mutated Tet repressor of the invention.

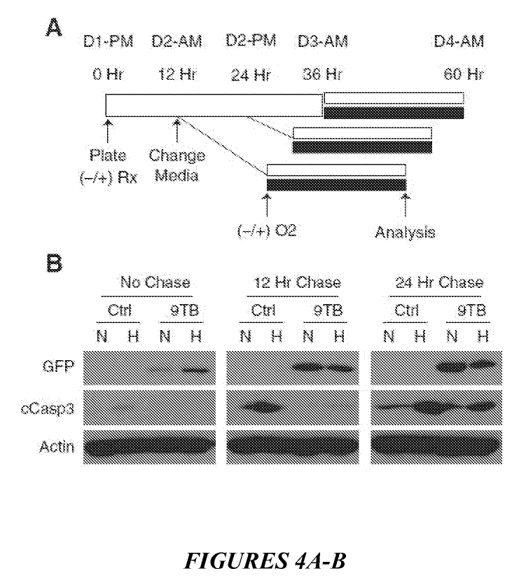

Additional mutated Tet repressors for incorporation into a fusion protein of the methods of the invention can be created according to same approach described above. A number of different classes of Tet repressors have been described, e.g., A, B, C, D and E (of which the Tn10 encoded repressor is a class B repressor). The amino acid sequences of the different classes of Tet repressors share a high degree of homology (i.e., 40-60% across the length of the proteins), including in the region encompassing the above-described mutations. The amino acid sequences of various classes of Tet repressors are shown and compared in U.S. Pat. No. 5,789,156 to Bujard et al. (see FIG. 4 thereof), and are also described in Tovar et al., “Identification and Nucleotide Sequence of the Class E Tet Regulatory Elements and Operator and Inducer Binding of the Encoded Purified Tet Repressor,” Mol. Gen. Genet. 215:76-80 (1988), each of which is hereby incorporated by reference in its entirety. Accordingly, equivalent mutations to those described above for the Tn10-derived Tet repressor can be made in other classes of Tet repressors for inclusion in a fusion protein of the invention. For example, amino acid position 95, which is an aspartic acid in all five repressor classes, can be mutated to asparagine in any class of repressor. Similarly, position 102, which is glycine in all five repressor classes, can be mutated to aspartic acid in any class of repressor. Additional suitable equivalent mutations will be apparent to those skilled in the art and can be created and tested for functionality by procedures described herein. Nucleotide and amino acid sequences of Tet repressors of the A, C, D and E classes are disclosed in Waters et al., “The Tetracycline Resistance Determinants of RP1 and Tn1721: Nucleotide Sequence Analysis,” Nucl. Acids Res. 11:6089-6105 (1983); Unger et al., “Nucleotide Sequence of the Gene, Protein Purification and Characterization of the pSC101-Encoded Tetracycline Resistance-gene-Repressor,” Gene 31(1-3):103-108 (1984); Unger et al., “Nucleotide Sequence of the Repressor Gene of the RA1 Tetracycline Resistance Determinant: Structural and Functional Comparison with Three Related Tet Repressor Genes,” Nucl. Acids Res. 12:7693-7703 (1984); and Tovar et al., “Identification and Nucleotide Sequence of the Class E Tet Regulatory Elements and Operator and Inducer Binding of the Encoded Purified Tet Repressor,” Mol. Gen. Genet. 215:76-80 (1988), each of which is hereby incorporated by reference in its entirety. These wild-type sequences can be mutated accordingly for use in the inducible regulatory system described herein.

Alternative to the above-described mutations, additional suitable mutated Tet repressors (e.g., having the desired functional properties described above) can be created by mutagenesis of a wild type Tet repressor and selection as described in U.S. Pat. No. 5,789,156 to Bujard et al. (see Example 1 thereof), which is hereby incorporated by reference in its entirety. The nucleotide and amino acid sequences of wild-type class B Tet repressors are disclosed in Hillen and Schollmeier, “Nucleotide Sequence of the Tn10 Encoded Tetracycline Resistance Gene,” Nucl. Acids Res. 11:525-539 (1983); Postle et al., “Nucleotide Sequence of the Repressor Gene of the TN10 Tetracycline Resistance Determinant,” Nucl. Acids Res. 12:4849-4863 (1984), each of which is hereby incorporated by reference in its entirety. References for the nucleotide and amino acid sequences of wild-type class A, C, D and E type repressors are cited above. A mutated Tet repressor can be created and selected, for example as follows: a nucleic acid (e.g., DNA) encoding a wild-type Tet repressor is subjected to random mutagenesis and the resultant mutated nucleic acids are incorporated into an expression vector and introduced into a host cell for screening. A screening assay, e.g., which allows for selection of a Tet repressor which binds to a Tet operator sequence only in the presence of a substituted tetracycline compound can be used. For example, a library of mutated nucleic acids in an expression vector can be introduced into an E. coli strain in which Tet operator sequences control the expression of a gene encoding a Lac repressor and the Lac repressor controls the expression of a gene encoding a selectable marker (e.g., drug resistance). Binding of a Tet repressor to Tet operator sequences in the host cell will inhibit expression of the Lac repressor, thereby inducing expression of the selectable marker gene. Cells expressing the marker gene are selected based upon the selectable phenotype (e.g., drug resistance). For wild-type Tet repressors, expression of the selectable marker gene will occur in the absence of tetracycline. A nucleic acid encoding a mutated Tet repressor may be selected using this system based upon the ability of the nucleic acid to induce expression of the selectable marker gene in the bacteria only in the presence of a substituted tetracycline compound.

Another approach for creating a mutated Tet repressor which binds to a class A tet operator is to further mutate the already mutated Tn10-derived Tet repressor described herein (a class B repressor) such that it no longer binds efficiently to a class B type operator but instead binds efficiently to a class A type operator. It has been found that nucleotide position 6 of class A or B type operators is the critical nucleotide for recognition of the operator by its complimentary repressor (position 6 is a G/C pair in class B operators and an A/T pair in class A operators) (see Wissman et al., “Saturation Mutagenesis of the Tn10-Encoded Tet Operator 01. Identification of Base-Pairs Involved in Tet Repressor Recognition,” J. Mol. Biol. 202:397-406 (1988), which is hereby incorporated by reference in its entirety). It has also been found that amino acid position 40 of a class A or class B Tet repressor is the critical amino acid residue for recognition of position 6 of the operator (amino acid position 40 is a threonine in class B repressors but is an alanine in class A repressors). It still further has been found that substitution of Thr40 of a class B repressor with Ala alters its binding specificity such that the repressor can now bind a class A operator (similarly, substitution of Ala40 of a class A repressor with Thr alters its binding specificity such that the repressor can now bind a class B operator) (see Altschmied et al., “A Threonine to Alanine Exchange at Position 40 of Tet Repressor Alters the Recognition of the Sixth Base Pair of Tet Operator from GC to AT,” EMBO J. 7:4011-4017 (1988), each of which is hereby incorporated by reference in its entirety). Accordingly, one can alter the binding specificity of the mutated Tn10-derived Tet repressor disclosed herein by additionally changing amino acid residue 40 from Thr to Ala by standard molecular biology techniques (e.g., site directed mutagenesis).

A mutated Tet repressor, e.g., having specific mutations at positions 71, 95, 101 and/or 102, as described above, can be created by introducing nucleotide changes into a nucleic acid encoding a wild-type repressor by standard molecular biology techniques, e.g. site directed mutagenesis or PCR-mediated mutagenesis using oligonucleotide primers incorporating the nucleotide mutations. Alternatively, when a mutated Tet repressor is identified by selection from a library, the mutated nucleic acid can be recovered from the library vector. To create a transcriptional regulator fusion protein of the invention, a nucleic acid encoding a mutated Tet repressor is then ligated in-frame to another nucleic acid encoding a transcriptional activation domain and the fusion construct is incorporated into a recombinant expression vector. The transcriptional regulator fusion protein can be expressed by introducing the recombinant expression vector into a host cell or animal.

The first polypeptide of the transactivator fusion protein is operatively linked to a second polypeptide which directly or indirectly activates transcription in eukaryotic cells. To operatively link the first and second polypeptides, typically, nucleotide sequences encoding the first and second polypeptides are ligated to each other in-frame to create a chimeric gene encoding a fusion protein. However, the first and second polypeptides can be operatively linked by other means that preserve the function of each polypeptide (e.g., chemically crosslinked). The second polypeptide of the transactivator may itself possess transcriptional activation activity (i.e., the second polypeptide directly activates transcription). The second polypeptide may also activate transcription by an indirect mechanism, through recruitment of a transcriptional activation protein to interact with the fusion protein.

Polypeptides which can function to activate transcription in eukaryotic cells are well known in the art. In particular, transcriptional activation domains of many DNA binding proteins have been described and have been shown to retain their activation function when the domain is transferred to a heterologous protein. A preferred polypeptide for use in the fusion protein of the methods of the invention is the herpes simplex virus virion protein 16 (referred to herein as VP16, the amino acid sequence of which is disclosed in Triezenberg et al., “Functional Dissection of VP16, the Trans-Activator of Herpes Simplex Virus Immediate Early Gene Expression,” Genes Dev. 2:718-729 (1988), which is hereby incorporated by reference in its entirety).

Other polypeptides with transcriptional activation ability in eukaryotic cells can be used in the fusion protein of the methods of the invention. Transcriptional activation domains found within various proteins have been grouped into categories based upon similar structural features. Types of transcriptional activation domains include acidic transcription activation domains, proline-rich transcription activation domains, serine/threonine-rich transcription activation domains and glutamine-rich transcription activation domains. Examples of acidic transcriptional activation domains include the VP16 regions already described and amino acid residues 753-881 of GAL4. Examples of proline-rich activation domains include amino acid residues 399-499 of CTF/NF 1 and amino acid residues 31-76 of AP2. Examples of serine/threonine-rich transcription activation domains include amino acid residues 1-427 of ITF 1 and amino acid residues 2-451 of ITF2. Examples of glutamine-rich activation domains include amino acid residues 175-269 of Oct I and amino acid residues 132-243 of Sp1. The amino acid sequences of each of the above described regions, and of other useful transcriptional activation domains, are disclosed in Seipel et al., “Different Activation Domains Stimulate Transcription From Remote (‘Enhancer’) and Proximal (‘Promoter’) Positions,” EMBO J. 13:4961-4968 (1992), which is hereby incorporated by reference in its entirety.

In addition to previously described transcriptional activation domains, novel transcriptional activation domains, which can be identified by standard techniques, are within the scope of the methods of the invention. The transcriptional activation ability of a polypeptide can be assayed by linking the polypeptide to another polypeptide having DNA binding activity and determining the amount of transcription of a target sequence that is stimulated by the fusion protein. For example, a standard assay used in the art utilizes a fusion protein of a putative transcriptional activation domain and a GAL4 DNA binding domain (e.g., amino acid residues 1-93). This fusion protein is then used to stimulate expression of a reporter gene linked to GAL4 binding sites (see, e.g., Seipel et al., “Different Activation Domains Stimulate Transcription From Remote (‘Enhancer’) and Proximal (‘Promoter’) Positions,” EMBO J. 13:4961-4968 (1992), which is hereby incorporated by reference in its entirety).

The second polypeptide of the fusion protein may indirectly activate transcription by recruiting a transcriptional activator to interact with the fusion protein. For example, a TetR or mutated TetR of the invention can be fused to a polypeptide domain (e.g., a dimerization domain) capable of mediating a protein-protein interaction with a transcriptional activator protein, such as an endogenous activator present in a host cell. It has been demonstrated that functional associations between DNA binding domains and transactivation domains need not be covalent (see, e.g., Fields and Song, “A Novel Genetic System to Detect Protein-Protein Interactions,” Nature 340:245-247 (1989); Chien et al., “The Two-Hybrid System: A Method to Identify and Clone Genes for Proteins that Interact with a Protein of Interest,” Proc. Natl. Acad. Sci. U.S.A. 31:9578-9582 (1991); Gyuris et al., “Cdi1, A Human G1 and S Phase Protein Phosphatase that Associates with Cdk2,” Cell 75:791-803 (1993); and Zervos, “Mxil, A Protein that Specifically Interacts with Max to Bind Myc-Max Recognition Sites,” Cell 72:223-232 (1993), each of which is hereby incorporated by reference in its entirety). Accordingly, the second polypeptide of the fusion protein may not directly activate transcription but rather may form a stable interaction with an endogenous polypeptide bearing a compatible protein-protein interaction domain and transactivation domain. Examples of suitable interaction (or dimerization) domains include leucine zippers (Landschulz et al., “The DNA Binding Domain of the Rat Liver Nuclear Protein C/EBP is Bipartite,” Science 243:1681-1688 (1989), which is hereby incorporated by reference in its entirety), helix-loop-helix domains (Murre et al., “Interactions Between Heterologous Helix-Loop-Helix Proteins Generate Complexes that Bind Specifically to a Common DNA Sequence,” Cell 58:537-544 (1989), which is hereby incorporated by reference in its entirety), and zinc finger domains (Frankel et al., “Tat Protein from Human Immunodeficiency Virus Forms a Metal-Linked Dimer,” Science 240:70-73 (1988), which is hereby incorporated by reference in its entirety). Interaction of a dimerization domain present in the fusion protein with an endogenous nuclear factor results in recruitment of the transactivation domain of the nuclear factor to the fusion protein, and thereby to a tet operator sequence to which the fusion protein is bound.

The agent that is capable of inducing the inducible transgene, and also has direct activity on a cell death or cell survival pathway, is preferably a tetracycline compound. The term “tetracycline” includes unsubstituted and substituted tetracycline compounds. The term “substituted tetracycline compound” includes derivatives or analogs of tetracycline compounds or compounds with a similar ring structure to tetracycline. Thus, the term “substituted tetracycline compound” includes tetracycline compounds with one or more additional substituents, e.g., at the 1, 2, 3, 4, 5, 6, 7, 8, 9, 10, 11, 11a, 12, 12a or 13 position or at any other position which allows the substituted tetracycline compound of the invention to perform its intended function, e.g., modulate gene expression or gene products.

Examples of unsubstituted tetracycline compounds such as minocycline, doxycycline, tetracycline, anhydrotetracycline, doxycycline, chlorotetracycline, oxytetracycline and others disclosed by Hlavka and Boothe, “The Tetracyclines,” In Handbook of Experimental Pharmacology 78, R. K. Blackwood et al. (eds.), Springer-Verlag, Berlin-New York, 1985; L. A. Mitscher, “The Chemistry of the Tetracycline Antibiotics”, Medicinal Research 9, Dekker, New York, 1978; Noyee Development Corporation, “Tetracycline Manufacturing Processes” Chemical Process Reviews, Park Ridge, N.J., 2 volumes, 1969; R. C. Evans, “The Technology of the Tetracyclines,” Biochemical Reference Series 1, Quadrangle Press, New York, 1968; and H. F. Dowling, “Tetracycline”, Antibiotic Monographs, no. 3, Medical Encyclopedia, New York, 1955, each of which is hereby incorporated by reference in its entirety.

Examples of substituted tetracycline compounds include, without limitation, chlortetracycline, oxytetracycline, demeclocycline, methacycline, sancycline, 9-tert-butyl-doxycycline, minocycline, tigecycline, chelocardin, rolitetracycline, lymecycline, apicycline, clomocycline, guamecycline, meglucycline, mepylcycline, penimepicycline, pipacycline, and etamocycline, penimocycline, 6-demethyl-6-deoxy-4-dedimethylaminotetracycline, tetracyclino-pyrazole, 7-chloro-4-dedimethylaminotetracycline, 4-hydroxy-4-dedimethylaminotetracycline, 12α-deoxy-4-dedimethylaminotetracycline, 5-hydroxy-6α-deoxy-4-dedimethylaminotetracycline, 4-dedimethylamino-12α-deoxyanhydrotetracycline, 7-dimethylamino-6-demethyl-6-deoxy-4-dedimethylaminotetracyc line, tetracyclinonitrile, 4-oxo-4-dedimethylaminotetracycline 4,6-hemiketal, 4-oxo-11a, C1-4-dedimethylaminotetracycline-4,6-hemiketal, 5a,6-anhydro-4-hydrazon-4-dedimethylamino tetracycline, 4-hydroxyimino-4-dedimethylamino tetracyclines, 4-hydroxyimino-4-dedimethylamino 5a,6-anhydrotetracyclines, 4-amino-4-dedimethylamino-5a,6 anhydrotetracycline, 4-methylamino-4-dedimethylamino tetracycline, 4-hydrazono-11a-chloro-6-deoxy-6-demethyl-6-methylene-4-dedi methylamino tetracycline, tetracycline quaternary ammonium compounds, anhydrotetracycline betaines, 4-hydroxy-6-methyl pretetramides, 4-keto tetracyclines, 5-keto tetracyclines, 5a, 11a dehydro tetracyclines, 11a Cl-6,12 hemiketal tetracyclines, 11a Cl-6-methylene tetracyclines, 6,13 diol tetracyclines, 6-benzylthiomethylene tetracyclines, 7,11a-dichloro-6-fluoro-methyl-6-deoxy tetracyclines, 6-fluoro (α)-6-demethyl-6-deoxy tetracyclines, 6-fluoro (β)-6-demethyl-6-deoxy tetracyclines, 6-α acetoxy-6-demethyl tetracyclines, 6-βacetoxy-6-demethyl tetracyclines, 7,13-epithiotetracyclines, oxytetracyclines, pyrazolotetracyclines, 11a halogens of tetracyclines, 12a formyl and other esters of tetracyclines, 5,12a esters of tetracyclines, 10,12a-diesters of tetracyclines, isotetracycline, 12-a-deoxyanhydro tetracyclines, 6-demethyl-12a-deoxy-7-chloroanhydrotetracyclines, B-nortetracyclines, 7-methoxy-6-demethyl-6-deoxytetracyclines, 6-demethyl-6-deoxy-5a-epitetracyclines, 8-hydroxy-6-demethyl-6-deoxy tetracyclines, monardene, chromocycline, 5a methyl-6-dimethyl-6-deoxy tetracyclines, 6-oxa tetracyclines, and 6-thia tetracyclines. Other derivatives and analogues comprising a similar four ring structure are also included. Tetracycline and several known tetracycline derivatives are well known in the art, and are identified in U.S. Patent Publication No. 2008/0118979 A1; U.S. Pat. Nos. 6,165,999; 5,834,450; 5,886,175; 5,567,697; 5,567,692; 5,530,557; 5,512,553; and 5,430,162; PCT Application Publ. Nos. WO 03/079984, WO 03/075857, WO 03/057169, WO 02/072545, WO 02/072532, WO 99/37307, WO 02/12170, WO 02/04407, WO 02/04406, WO 02/04404, WO 01/98260, WO 01/98259, WO 01/98236, WO 01/87824, WO 01/74761, WO 01/52858, WO 01/19784, WO 84/01895, each of which is hereby incorporated by reference in its entirety. Other examples of substituted tetracycline compounds are described in EP 0582810 B1; EP 0536 515B1; EP 0582 789B1; EP 0582 829B1; EP 0582788B1; U.S. Pat. No. 5,530,117; U.S. Pat. No. 5,495,030; U.S. Pat. No. 5,495,018; U.S. Pat. No. 5,494,903; U.S. Pat. No. 5,466,684; EP 0535 346B1; U.S. Pat. No. 5,457,096; U.S. Pat. No. 5,442,059; U.S. Pat. No. 5,430,162; U.S. Pat. No. 5,420,272; U.S. Pat. No. 5,401,863; U.S. Pat. No. 5,401,729; U.S. Pat. No. 5,386,041; U.S. Pat. No. 5,380,888; U.S. Pat. No. 5,371,076; EP 618 190; U.S. Pat. No. 5,326,759; EP 582 829; EP 528 810; EP 582 790; EP 582 789; EP 582 788; U.S. Pat. No. 5,281,628; EP 536 515; EP 535 346; WO 96/34852; WO 95/22529A1; U.S. Pat. No. 4,066,694; U.S. Pat. No. 3,862,225; U.S. Pat. No. 3,622,627; WO 01/87823A1; WO 00/28983A1; WO 07/014,154; WO 06/084,265; WO 06/047,671; WO 06/047,756; US 2006-0287283; WO 06/549,717; and US 2005-0143352, each of which is hereby incorporated by reference in its entirety.

As noted above, the transgene of the present invention (and the transcriptional activator protein) are intended to be expressed in a cell. The cell is a “host cell” in that it does not naturally contain both the transgene and transcriptional activator protein. The host cell can be either a eukaryotic or prokaryotic cell, whether a primary cell isolate or a propagated cell line.

Non-limiting examples of mammalian cell lines which can be used include, without limitation, COS (e.g., ATCC No. CRL 1650 or 1651), BHK (e.g., ATCC No. CRL 6281), CHO (e.g., ATCC No. CCL 61), HeLa (e.g., ATCC No. CCL 2), 293 (ATCC No. 1573), CHOP, and NS-1 cells.

Other types of cells that are of interest include, without limitation, cancer cells, neuronal cells, myocytes, or any other somatic cell that exhibits selective vulnerability to either pharmacological or physiological toxins. Thus, the cells can be isolated from solid tumors found in all non-hematopoietic sites, including, but not limited to lung, breast, colon and entire gastrointestinal tract, prostate, brain, pancreas, and skin. The cells can also include non-solid tumors, e.g., lymphomas, leukemias, and plasma cell neoplasms, and involves the analysis of patient tissues that include all hematopoietic organs, without limitation, blood, lymph nodes, tonsil, spleen, thymus, and bone marrow. The invention may also be used with cancer cell lines, including but not limited to the human prostate cancer cell lines DU-145, PC-3, LNCaP, PPC-1, and TSU-Prl, the MBT-2 bladder cancer cell line, and the non-small cell lung cancer cell line KNS62. Exemplary neuronal cells and cell lines, including brain, spinal cord, and peripheral ganglia, as well as other specialized neurons such as sensory neurons, motor neurons, and interneurons. Other examples of unique neuronal types include, but are not limited to, basket cells, betz cells, medium spiny neurons, purkinje cells, pyramidal cells, renshaw cells, granule cells, and anterior horn cells. Myocytes that can be used include, without limitation, human cardiac myocytes, human skeletal myocytes, and human smooth muscle cells.

Regardless of the type of host cell to be used, the host cell is transformed or transfected to include the transgene and transcriptional activator protein. The terms “transformation” and “transfection” refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2d ed., Cold Spring Harbor Laboratory, Cold Spring Harbor Laboratory Press, Cold Spring Harbor, N.Y., 1989), which is hereby incorporated by reference in its entirety.

As noted above, the inducible transgene is capable of expressing a gene product that has activity on a cell death or cell survival pathway. According to one embodiment, the transgene expression product activates a cell death pathway (to promote cell death). According to another embodiment, the transgene expression product activates a cell survival pathway (to promote cell survival).

One of the principle advantages of an inducible gene expression system is having the ability to generate stable transfectants harboring toxic genes where conventional constitutive gene expression systems might otherwise fail (Marchetti et al., “Inducible Expression of a MAP Kinase Phosphatase-3-GFP Chimera Specifically Blunts Fibroblast Growth and Ras-Dependent Tumor Formation in Nude Mice,” J. Cell Physiol. 199(3):441-450 (2004) and Shoshani et al., “Identification of a Novel Hypoxia-Inducible Factor 1-Responsive Gene, RTP801, Involved in Apoptosis,” Mol. Cell. Biol. 22(7):2283-2293 (2002), each of which is hereby incorporated by reference in its entirety).

According to one embodiment, the transgene expression product is a nucleic acid molecule, examples of which include, without limitation, RNAi and aptamers.

RNAi refers to interfering RNA. RNAi is well-known in the field to be effected by siRNA, miRNA, shRNA and other RNAi inducing agents. In this specification, it will be understood that the terms siRNA and RNAi are interchangeable. Although siRNA will be referred to in general in the specification, it will be understood that any other RNA inducing agent may be used, including shRNA, miRNA, or another RNAi-inducing vector whose presence within a cell results in production of an siRNA or shRNA targeted to inhibit expression of a particular target protein that is involved in regulating a cell death or cell survival pathway. An important feature of RNAi affected by siRNA is the double stranded nature of the RNA and the absence of large overhanging pieces of single stranded RNA, although dsRNA with small overhangs and with intervening loops of RNA has been shown to effect suppression of a target gene.

RNA interference is a multistep process and is generally activated by double-stranded RNA (dsRNA) that is homologous in sequence to the targeted gene. Introduction of long dsRNA into the cells of organisms leads to the sequence-specific degradation of homologous gene transcripts. The long dsRNA molecules are metabolized to small (e.g., 21-23 nucleotide (nt)) interfering RNAs (siRNAs) by the action of an endogenous ribonuclease known as Dicer. The siRNA molecules bind to a protein complex, termed RNA-induced silencing complex (RISC), which contains a helicase activity and an endonuclease activity. The helicase activity unwinds the two strands of RNA molecules, allowing the antisense strand to bind to the targeted claudin-1 RNA molecule. The endonuclease activity hydrolyzes the RNA at the site where the antisense strand is bound. Therefore, RNAi is an antisense mechanism of action, as a single stranded (ssRNA) RNA molecule binds to the target RNA molecule and recruits a ribonuclease that degrades the target RNA.

Examples of RNAi targets that can modulate cell survival pathways include, without limitation, members of the BH3 family of cell death modulators such as Bax (NCBI Ref. Seq. No. NP_{—}004315.1; NM_{—}004324.3), Bid (NCBI Ref. Seq. No. NP_{—}001187.1; NM_{—}001196.2), Bim (NCBI Ref. Seq. No. NP_{—}001191035.1; NM_{—}001204106.1), and Bnip3 (NCBI Ref. Seq. No. NP_{—}004043.2; NM_{—}004052.2); cysteine proteases such as caspase 2 (NCBI Ref. Seq. No. NP_{—}001215.1; NM_{—}001224.4), caspase 3 (NCBI Ref. Seq. No. NP_{—}004337.2; NM_{—}004346.3), caspase 7 (NCBI Ref. Seq. No. NP_{—}001218.1; NM_{—}001227.3), caspase 8 (NCBI Ref. Seq. No. NP_{—}001073593.1; NM_{—}001080124.1), caspase 9 (NCBI Ref. Seq. No. NP_{—}001220.2; NM_{—}001229.3), and caspase 12 (NCBI Ref. Seq. No. NP_{—}001177945.1; NM_{—}001191016.1); pro-apoptotic nucleases such as EndoG (NCBI Ref. Seq. No. NP_{—}004426.2; NM_{—}004435.2)); PARP (NCBI Ref. Seq. No. NP_{—}006428.2; NM_{—}006437.3); and other caspase-3 targets, transcription factors regulating cell death pathways such as p53 (NCBI Ref. Seq. No. NP_{—}000537.3; NM_{—}000546.4), HIF-1a (NCBI Ref. Seq. No. NP_{—}001521.1; NM_{—}001530.3), NFkB (NCBI Ref. Seq. No. NP_{—}001158884.1; NM_{—}001165412.1), cJun (NCBI Ref. Seq. No. NP_{—}002219.1; NM_{—}002228.3.), and CHOP-10 (NCBI Ref. Seq. No. NP_{—}001181986.1; NM_{—}001195057.1); effector proteins activated under conditions of ER stress such as ATF4 (NCBI Ref. Seq. No. NP_{—}001666.2; NM_{—}001675.2.), ATF6 (NCBI Ref. Seq. No. NP_{—}031374.2; NM_{—}007348.2.), IRE-1 (NCBI Ref. Seq. No. NP_{—}001424.3; NM_{—}001433.3), CHOP-10 (NCBI Ref. Seq. No. NP_{—}001181986.1; NM_{—}001195057.1), and PKR (NCBI Ref. Seq. No. NP_{—}001129123.1; NM_{—}001135651.1); lysosomal enzymes as well as other stress activated proteases involved in ischemic signaling such as cathepsins (e.g., NCBI Ref. Seq. No. NP_{—}003784.2; NM_{—}003793.3.), calpains (e.g., NCBI Ref. Seq. No. NP_{—}001003962.1; NM_{—}001003962.1.); cellular proteins with either kinase or phosphatase function such as Akt (NCBI Ref. Seq. No. NP_{—}001014431.1; NM_{—}001014431.1), PTEN (NCBI Ref. Seq. No. NP_{—}000305.3; NM_{—}000314.4), and MKP-1 (NCBI Ref. Seq. No. NP_{—}004408.1; NM_{—}004417.3.); mitochondrial proteins involved in apoptotic signaling pathways including AIF (NCBI Ref. Seq. No. NP_{—}001614.3; NM_{—}001623.3.), cytochrome C 9 (NCBI Ref. Seq. No. NP_{—}001907.2; NM_{—}001916.3.), etc.), and factors involved in either autophagic and mitophagic signaling cascades. Each of the above-identified Genbank accessions is hereby incorporated by reference in its entirety. RNAi molecules against these targets can be designed using the online design tools provided by Ambion (Life Technologies, Inc.), Clontech, and others. Alternatively, DNA constructs encoding RNAi molecules against these targets are commercially available from SantaCruz Biotechnology, Inc. and other RNAi vendors.

The term aptamers includes short strands of oligonucleotides that bind to specific target molecule and modulate its activity. Aptamers are single-stranded, partially single-stranded, partially double-stranded, or double-stranded nucleotide sequences, advantageously a replicatable nucleotide sequence, capable of specifically recognizing a selected non-oligonucleotide molecule or group of molecules (in this case, one or more target proteins that are active in cell death or cell survival pathways) by a mechanism other than Watson-Crick base pairing or triplex formation. Aptamers include, without limitation, defined sequence segments and sequences comprising nucleotides, ribonucleotides, deoxyribonucleotides, nucleotide analogs, modified nucleotides and nucleotides comprising backbone modifications, branchpoints and normucleotide residues, groups or bridges. Aptamers include partially and fully single-stranded and double-stranded nucleotide molecules and sequences; synthetic RNA, DNA, and chimeric nucleotides; hybrids; duplexes; heteroduplexes; and any ribonucleotide, deoxyribonucleotide, or chimeric counterpart thereof and/or corresponding complementary sequence, promoter, or primer-annealing sequence needed to amplify, transcribe, or replicate all or part of the aptamer molecule or sequence.

Nucleic acid aptamers include monovalent aptamers as well as multivalent aptamers (e.g., aptamers that are bivalent, trivalent, tetravalent, pentavalent, etc.). Methods of making bivalent and multivalent aptamers and their expression in multi-cellular organisms are described in U.S. Pat. No. 6,458,559 to Shi et al., which is hereby incorporated by reference in its entirety. A method for modular design and construction of multivalent nucleic acid aptamers, their expression, and methods of use are described in U.S. Patent Publication No. 2005/0282190 to Shi et al., which is hereby incorporated by reference in its entirety. Aptamers may be designed to modulate the activity of cell death or cell survival target molecules. Identifying suitable, selective aptamer molecules can be carried out using the well known SELEX (Systematic Evolution of Ligands by Exponential Enrichment) procedure. SELEX is an evolutionary, in vitro combinatorial chemistry process used to identify aptamers binding to a ligand or target from large pools of diverse oligonucleotides. SELEX is an excellent system for isolating aptamers from a random pool under specific customizable binding conditions. The SELEX process has provided an alternative for generating single stranded DNA or RNA oligonucleotides that bind tightly and specifically to given ligands or targets. (Tuerk et al., “Systematic Evolution of Ligands by Exponential Enrichment: RNA Ligands to Bacteriophage T4 DNA Polymerase,” Science 249:505-510 (1990); Ellington A., “RNA Selection. Aptamers Achieve the Desired Recognition,” Curr. Biol. 4:427-429 (1994); Ellington et al., “In vitro Selection of RNA Molecules that Bind Specific Ligands,” Nature 346:818-822 (1990), each of which is hereby incorporated by reference in its entirety). SELEX experiments have been exploited to investigate the functional and structural aspects of nucleic acids, and the identified aptamers have become an important tool for the research of molecular diagnostics, molecular recognition, molecular biology, and molecular evolution (Uphoff et al., “In vitro Selection of Aptamers: The Dearth of Pure Reason,” Curr. Opin. Struct. Biol. 6:281-288 (1996), which is hereby incorporated by reference in its entirety).

According to another embodiment, the transgene expression product is a protein or polypeptide. Examples of proteins or polypeptides that moderate cell survival/death pathways include, without limitation, limitation, members of the BH3 family of cell death modulators (i.e., Bax, Bid, Bim, Bnip3), cysteine proteases (i.e., caspases 2, 3, 7, 8, 9 and 12), pro-apoptotic nucleases (e.g., EndoG), PARP and other caspase-3 targets, transcription factors regulating cell death pathways (i.e., p53, HIF-1a, NFkB, cJun, CHOP-10), effector proteins activated under conditions of ER stress (ATF4, ATF6, IRE-1, CHOP-10, PKR), lysosomal enzymes as well as other stress activated proteases involved in ischemic signaling (e.g., cathepsins, calpains, etc.), cellular proteins with either kinase or phosphatase function (e.g., Akt, PTEN, MKP-1), mitochondrial proteins involved in apoptotic signaling pathways (AIF, cytochrome C, etc.), and factors involved in either autophagic and mitophagic signaling cascades (see supra for applicable NCBI Ref. Seq. Nos.).

The transgene product and the agent can have activity on the same cell death or cell survival pathway, or they can have activity on different cell death or cell survival pathways.

Tet-regulated gene expression systems are being used more commonly for commercial applications including the large-scale production of therapeutically useful biologicals and drug screening. The present invention has particularly important implications for the latter setting, where tightly regulated stable cell line systems are required for compound drug screening campaigns to identify pro- and anti-apoptotic compounds for cancer and neurodegenerative conditions. Based on the successful demonstration in the accompanying examples, it is expected that the protocol modifications will be easily scaled for such high throughput applications. While these studies were performed using an in vitro culture system, 9-TB has been used successfully in vivo to study the dynamic behavior of tet-responsive transgenic sequences in the central nervous system. Thus, given the recent advances in the fields of cell-based and viral-based therapeutics for neurodegenerative diseases and stroke, having access to a drug that can both stimulate therapeutic gene expression and limit the activation of endogenous apoptotic programs represents a novel opportunity for therapeutic synergy not previously recognized.

As noted above, the step of exposing the cell to the agent is carried out in a manner effective to induce expression of the transgene while substantially minimizing direct activity of the inducing agent. The exposing of the cell to the agent is preferably carried out according to a pulse-washout schedule, which achieves both of these objectives. During the “pulse” phase, the cell is exposed to the agent. Because the agent possesses a “half-life,” the agent will still be present in the cell following cessation of the “exposing” although its concentration within the cell will diminish over time during the “washout” phase of the cycle. The pulse-washout cycle can be repeated, as desired, using the same or a varying pulse phase or the same or a varying washout phase.

The pulse-washout schedule exposes the cell to the agent for an amount of time that is generally insufficient to allow for direct activation of a cell death or cell survival pathway.

According to one embodiment, the minimum amount of pulse time is about 2 hours and the maximum amount of pulse time is about 12 hours. The pulse exposes the cell for up to a maximum of about 12 hours, or up to about 10 hours, or up to about 8 hours, or up to about 6 hours, or up to about 4 hours, or up to about 2 hours. Exposure for between about 4 hours and about 8 hours is preferred.

According to one embodiment, the washout period is sufficient to reduce the intracellular concentration of the agent (due to its “half-life”) while at the same time allowing for transactivation of transgene expression. The washout period is preferably maintained for at least about 24 hours, more preferably at least about 36 hours, most preferably at least about 48 hours. The washout period should maintain a low enough concentration of the agent to promote extended expression of the transgene, while substantially minimizing direct activity of the agent on the cell death or cell survival pathway. In certain embodiments, the washout period allows for transgene expression for up to about 60 hours.

The pulse-washout schedule can be used once or it can be repeated in a particular host cell.

The pulse-washout schedule can also be accompanied by exposing the cell to an additional agent that can act on the transgenic host cell. The other agent can be used to assess its ability to enhance or inhibit effects of the transgene expression product on the cell death or cell survival pathway. Examples of suitable agents include, without limitation, chemotherapeutic agents, radiotherapeutic agents, kinase or phosphatase inhibitors, antiseizure medications, anti-inflammatory agents or other immunomodulators, soluble growth factors including peptide analogs that regulate receptor mediated signal transduction pathways, and scavengers of cellular reactive oxygen species. The additional agent can be introduced to the cell simultaneously with or serially with (i.e., either before or after) transgene induction.

In one embodiment, the host cell is present in an ex vivo culture. In this embodiment, the pulse phase of the schedule is carried out by introducing the agent into a cell culture medium at a concentration suitable to achieve transgene induction, and the washout phase is carried out by removing the agent from the culture medium, typically by replacing the culture medium.

In one embodiment, the host cell is present in an individual, preferably though not exclusively a mammalian organism. In certain embodiments, the mammalian organism is model organism for a disease state involving cell survival or cell death pathways, e.g., cancer, stroke, aging, myocardial infarction, global ischemic injury after cardiac arrest and other organ injury induced by tissue ischemia. In this embodiment, the pulse phase of the schedule is carried out by administering the agent into the organism at a concentration and rate suitable to achieve transgene induction while at the same time, considering the “half-life”, at a rate and dosage suitable to allow for washout to be carried out by cessation of administration.

A further aspect of the present invention relates to a kit that includes a response plasmid comprising (i) a promoter-effective nucleic acid molecule that includes a tetracycline-responsive element (TRE) and (ii) an insertion site suitable for introduction of an open reading frame of interest. The kit includes a regulator plasmid comprising a transgene that encodes a fusion protein comprising a first polypeptide that binds to the TRE in the presence or absence of a substituted tetracycline compound and is operatively linked to a second polypeptide that regulates transcription in cells. The kit preferably includes instructions for preparing a transgenic cell that can express the open reading frame of interest and the fusion protein, and exposing the transgenic cell to a substituted tetracycline compound according to a schedule that is effective to induce expression of the open reading frame of interest while substantially minimizing direct activity of the substituted tetracycline compound on a cell death or cell survival pathway. The kit may also contain one or more of the following: the substituted tetracycline compound and/or a cell line.

In other embodiments, the kit contains one or more than one host cell that comprises an inducible transgene that expresses a gene product having activity on a cell death or cell survival pathway, and one or more than one agent that is capable of inducing the inducible transgene (but also has direct activity on a cell death or cell survival pathway). In certain embodiments, a suite of host cells can be provided, representing different source tissues or different cancer cell lines, or having different transgenes capable of expressing different expression products of the type described above. These host cells can be used to assess the efficacy of the one or more transgene expression products in combination with other agents administered simultaneously with or serially with (i.e., either before or after) transgene induction.

### EXAMPLES

The following examples are provided to illustrate embodiments of the present invention but they are by no means intended to limit its scope.

### Materials and Methods

Reagents—

Doxycycline (4-[Dimethylamino]-1,4,4a,5,5a,6,11,12a-octahydro-3,5,10,12,12apentahydroxy-6-methyl-1,11-dioxo-2-naphthacenecarboxamide (“Dox”); Sigma-Aldrich, St. Louis, Mo., United States) and the derivative 9-t-butyl Dox (9-TB; a kind gift from Dr. Mark Nelson, Paratec Pharmaceuticals, Boston, Mass.) were resuspended to a final concentration of 10 mg/ml in water (Nelson et al., “Versatile and Facile Synthesis of Diverse Semisynthetic Tetracycline Derivatives Via Pd-Catalyzed Reactions,” J. Org. Chem. 68:5838-5851 (2003), which is hereby incorporated by reference in its entirety). Because of its structural modifications, 9TB is much more hydrophobic/lipophylic than other related compounds, shows a 10-fold higher binding affinity on the tet repressor relative to tetracycline, and is 10-times more potent compared to Dox in activating transcription from the rtTA (Zhu et al., “Silencing and Un-silencing of Tetracycline-Controlled Genes in Neurons,” PLoS One 2(6):e533 (2007), which is hereby incorporated by reference in its entirety). Hygromycin was obtained from Invitrogen (Carlsbad, Calif.).

Cloning and Stable Cell Line Production—

Inducible GFP expression was achieved by sub-cloning the IRES-GFP fragment into the tetracycline responsive bi-directional vector pBIG2i kindly provided by Dr. Craig Strathdee (Amgen, Inc., Cambridge, Mass.). Strathdee et al., “Efficient Control of Tetracycline-Responsive Gene Expression From an Autoregulated Bi-Directional Expression Vector,” Gene 229(1-2):21-29 (1999), which is hereby incorporated by reference in its entirety). This construct, designated pBig2i-MCS-IRES-GFP, was initially tested for inducible transgene expression by transfection into HN33 cells. The HN33.11 line (a gift from Bruce Wainer, Emory, Ga.) was passaged in Dulbecco's modified Eagle's medium (DMEM)/high glucose (HG)/5% stripped serum used to minimize exposure to tetracyclines, which may interfere with transgene inducibility (Innovative Research, Novi, Mich.). HN33 cells were generated by crossing mouse hippocampal neurons with the immortalized N18TG2 line as described (Lee et al., “Immortalized Young Adult Neurons From the Septal Region: Generation and Characterization,” Dev. Brain Res. 52(1-2):219-228 (1990) and Lee et al., “Neuronal Properties and Trophic Activities of Immortalized Hippocampal Cells From Embryonic and Young Adult Mice,” J. Neurosci. 10:1779-1787 (1990), which are hereby incorporated by reference in their entireties). HN33 cells maintain neuronal properties and have been used to study neuronal responses to hypoxic stress (Jin et al., “Vascular Endothelial Growth Factor: Direct Neuroprotective Effect in in vitro Ischemia,” Proc. Nat'l Acad. Sci. 97(18):10242-10247 (2000); Keilhoff et al., “Minocycline Protects Schwann Cells from Ischemia-Like Injury and Promotes Axonal Outgrowth in Bioartificial Nerve Grafts Lacking Wallerian Degeneration,” Exp. Neurol. 212:189-200 (2008); Lamartina et al., “Stringent Control of Gene Expression In vivo by Using Novel Doxycycline-Dependent Trans-Activators,” Hum. Gene Ther. 13:199-210 (2002); Lee et al., “Neuronal Properties and Trophic Activities of Immortalized Hippocampal Cells from Embryonic and Young Adult Mice,” J. Neurosci. 10:1779-1787 (1990); Lee et al., “Immortalized Young Adult Neurons from the Septal Region: Generation and Characterization,” Brain Res. Dev. Brain Res. 52:219-228 (1990); Marchetti et al., “Inducible Expression of a MAP Kinase Phosphatase-3-GFP Chimera Specifically Blunts Fibroblast Growth and ras-Dependent Tumor Formation in Nude Mice,” J. Cell Physiol. 199:441-450 (2004); Nelson et al., “Versatile and Facile Synthesis of Diverse Semisynthetic Tetracycline Derivatives via Pd-Catalyzed Reactions,” J. Org. Chem. 68:5838-5851 (2003); and Paillard, “‘Tet-On’: A Gene Switch for the Exogenous Regulation of Transgene Expression,” Hum. Gene Ther. 9:983-985 (1998); Schmidt-Kastner et al., Neuroglobin mRNA expression after transient global brain ischemia and prolonged hypoxia in cell culture,” Brain Res. 1103(1):173-180 (2006), which are hereby incorporated by reference in their entireties). HN33 stable lines were generated by batch transfection and selection with hygromycin according to standard protocols maintained in DMEM/8% stripped serum/supplemented with 200 μg/ml hygromycin for maintenance. Stripped serum was used throughout to minimize basal pBig2i activity (Innovative Research, Novi, Mich.).

In Vitro Hypoxia Model—

The HN33.11 cell line initially derived through the immortalization of mouse hippocampal neurons, exhibits many neuronal properties including membrane excitability and have been used to model apoptotic responses previously (Shi et al., “Involvement of Platelet-Activating Factor in Cell Death Induced Under Ischemia/Postischemia-Like Conditions in an Immortalized Hippocampal Cell Line,” J. Neurochem. 70:1035-1044 (1998), which is hereby incorporated by reference in its entirety). In vitro hypoxia was achieved using a triple gas incubator (Binder, Marietta, Ohio) and oxygen tension was monitored via an on-board zirconium oxide sensor, which exhibits linear sensing proportional to oxygen concentration. Cells were exposed to 0.5% O₂ for 18 hours unless otherwise specified.

Western Blotting—

Cell lysates were obtained by rinsing monolayers with ice-cold PBS×1 followed by the addition of RIPA buffer containing protease inhibitors (Sigma-Aldrich, St. Louis, Mo.). Samples were boiled in Laemmeli buffer for 1 minute and electrophoresed under reducing conditions and resolved by electrophoresis on polyacrylamide gels. Proteins were transferred to PVDF membranes and blocked in TBS-T (50 nM Tris-HCl, pH 8.0, 0.9% NaCl, and 0.1% Tween-20) containing 5% non-fat dry milk for 1 hour at room temperature. Antibodies used in this study included: GFP (1:2000; Novus Biologicals, NB-100-131, Littleton, Colo.), and cleaved caspase-3 (1:1000; CST, Danvers, Mass.), and actin (1:10 000; Santa Cruz Biologicals, CA). Blots were exposed to the appropriate secondary antibodies conjugated with HRP (1:5000; Santa Cruz Biologicals, CA) prior to chemiluminescent detection (ECL Reagent, Amersham/GE Life Sciences, Piscataway, N.J.). Densitometry was performed using 8-bit TiFF files and the Java-based application ImageJ. The area under the sample curves was determined using the analyze gel function, setting the relative background intensities with the line tool. The fold-activation reflects the level of hypoxia-induced caspase-3 cleavage compared to normoxic samples (n=2 for each comparison group).

### Example 1

### Comparison of the Dose Response Behavior of the Inducible GFP Reporter Line to a Range of Dox and 9-TB Concentrations

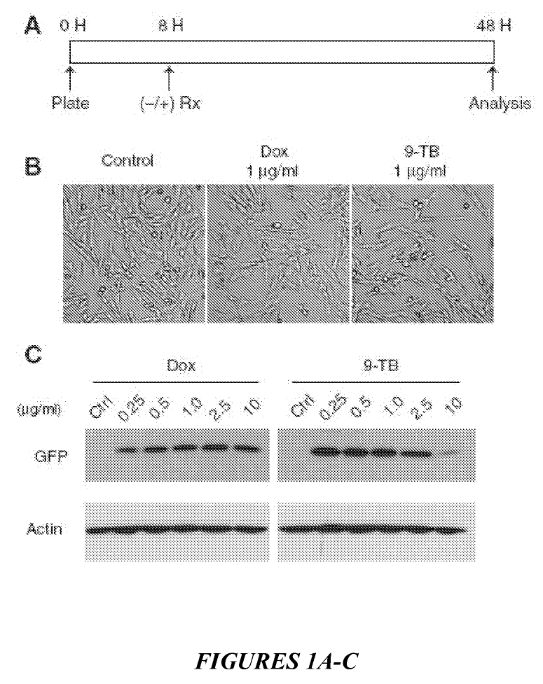

FIG. 1C is a comparison of the dose response behavior of the inducible GFP reporter line to a range of Dox and 9-TB concentrations. After plating, cells were exposed to either drug (0.25-10 μg/ml) continuously for 40 hours prior to analysis (FIG. 1A). Neither drug produced clear effects on cell morphology at intermediate concentrations (FIG. 1B). As expected, 9TB stimulated higher levels of GFP expression from the TetO promoter relative to Dox exposure, likely due to its hydrophobic properties that support 10-fold higher binding affinity to the tet-repressor (Zhu et al., “Silencing and Un-silencing of Tetracycline-Controlled Genes in Neurons,” PLoS One 2(6):e533 (2007), which is hereby incorporated by reference in its entirety). Lower levels of GFP expression observed with high-dose 9-TB (10 μg/ml) were associated with slight decrease in actin levels (FIG. 1C) and changes in the growth and morphology of cultured cells.

### Example 2

### Tetracycline Compounds Inhibit Hypoxia-Induced Caspase-3 Cleavage

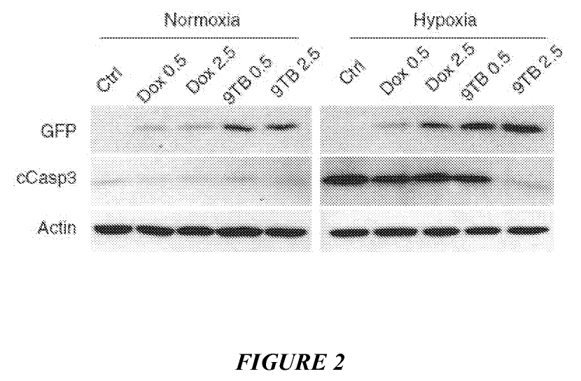

FIG. 2 shows the results of exposing the cultures to either tet-analog resulting in the expected pattern of GFP expression. In addition to exhibiting the morphological and electrophysiological features similar to their parental hippocampal neurons (Lee et al., “Neuronal Properties and Trophic Activities of Immortalized Hippocampal Cells From Embryonic and Young Adult Mice,” J. Neurosci. 10:1779-1787 (1990), which is hereby incorporated by reference in its entirety), the HN33.11 somatic fusion cell line also remains sensitive to hypoxic challenge. In vitro hypoxia (0.5% O2, 24 hours) induces a marked increase in levels of activated, cleaved caspase 3, which is a marker of apoptotic signaling in mammalian cells (FIG. 2, control normoxia vs. hypoxia). However, while cultures exposed to either tet-analog resulted in the expected pattern of GFP expression, continuous exposure to either Dox or 9-TB inhibited hypoxia-induced caspase-3 cleavage between 50-100% (FIG. 2). Thus, in addition to stimulating transgene expression, ligand treatment produces undesirable off-target effects by directly inhibiting apoptotic signaling. This confounder poses a particular problem when studying genes with putative pro- or anti-apoptotic effects.

### Example 3

### Pulse Dosing with 9-TB Induces Transgene Expression and Limits Effects on Hypoxia-Induced Caspase-3 Cleavage

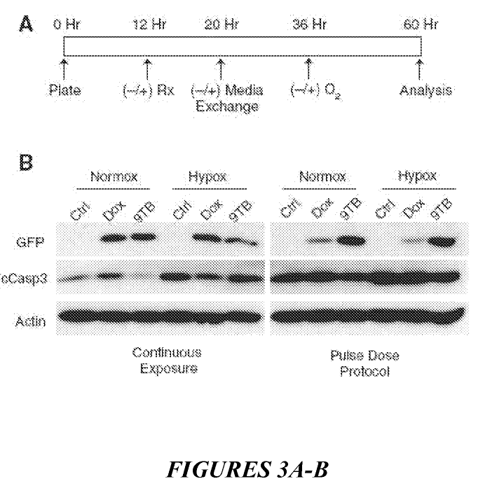

Based on the observed interference by Dox and 9-TB with the effects of transgene expression, the ability of pulse dosing to overcome the problem was explored. FIG. 3A is a diagram of an approach using overnight drug exposure (12 hours) to trigger gene expression, followed by a washout period (16 hours) accomplished by full-volume replacement of the culture media prior to hypoxic injury. Tet-regulated gene expression generally exhibits delayed kinetics both in terms of gene induction after drug exposure as well as with transgene silencing upon removal of ligand.

FIG. 3B is a comparison of GFP continuous exposure versus pulse dose protocol. Comparing the effects of pulsed Dox or 9-TB (2.5 μg/ml) against continuous drug exposure, it was found that while pulse dosing with Dox lowered GFP transgene activation compared to chronic exposure, pulse dosing with 9-TB produced dramatically increased expression levels (FIG. 3B, GFP continuous vs. pulse). As expected, both drugs interfered with caspase-3 cleavage. Despite higher overall levels of caspase activity after pulse-dosing, the modified protocol ameliorated dox-mediated suppression of hypoxia-induced caspase-3 cleavage seen after continuous ligand exposure (FIG. 3B). This effect was less impressive in 9-TB pulse-dosed samples (FIG. 3B, right panel, lanes 4 vs. 6).

### Example 4

### Comparison of the Effect of the Washout Period on Caspase-3 Cleavage Profiles

Based on the dramatic success with dox and the partial success with 9-TB in Example 3, an alternative schedule was explored to balance transgene expression with the absence of cCasp-3 interference. To improve the ratio of transgene expression and caspase-3 cleavage following hypoxic exposure, the effect of varying the washout period would have on GFP and cCasp-3 levels in 9-TB (2.5 μg/ml) induced cultures was tested (FIG. 4A). Shorter chase periods were associated with lower levels of transgene expression. FIG. 4B illustrates the differences in results between no chase period, a 12-hour chase period, and a 24-hour chase period. Results indicate that 9TB's suppressive effects on caspase-3 cleavage were augmented by shorter washout periods with a sharp decline occurring between the 12 and 24-hour chase periods (FIG. 4B).

### Example 5

### Optimization of Duration of 9-TB Pulse to Maximize Transgene Expression

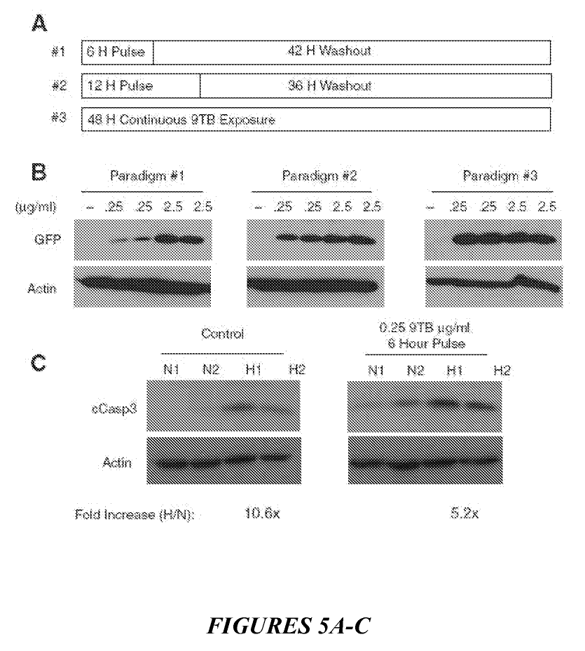

To further optimize the pulse-washout protocol, additional alternative schedules were explored to balance transgene expression with the absence of cCasp-3 interference. FIG. 5A illustrates the dosages for two dosing periods (6 hours and 12 hours) and extended incubation for 48 hours. FIG. 5B is a comparison of the effect of varying 9-TB dose and the duration of pulse exposure on transgene induction. Continuous dosing with 0.25 and 2.5 μg/ml 9-TB resulted in nearly equivalent transgene induction after 48-hours exposure (FIG. 5B, panel C). While clear differences were observed between 6 and 12-hour exposures at the lower 9-TB dose (0.25 μg/ml), treatment with 2.5 μg/ml produced equivalent levels of transgene expression. Taken together, these results demonstrate that brief drug exposure (6 hours) followed by a prolonged washout period (>24 hours) best optimizes the ratio of transgene induction and hypoxia-induced caspase cleavage in the stable tet-ON cell line. FIG. 5C shows the effects of low dose 9-TB on caspase processing relative to untreated controls.

### Discussion of Examples 1-5

In addition to their potent antimicrobial properties, tetracycline analogs also have protective effects on eukaryotic cells in part through effects on inflammatory and apoptotic signaling cascades. In the course of analyzing the effects of immediate early gene expression after in vitro hypoxia, it was discovered that in addition to inducing rtTA-regulated expression from a TetO-cytomegalovirus (CMV) promoter, continuous dox exposure inhibited apoptotic signaling as measured by cumulative levels of caspase-3 cleavage. This unintended off-target effect represented a technical hurdle that precluded studying the influence of hypoxia-induced transcription on cell injury. The preceding Examples demonstrate that a relatively brief pulse-dosing protocol using the dox analog 9-TB can support tet-on inducible gene expression without blocking endogenous apoptotic signaling cascades typically seen when using continuous dosing regimens and common ligands. This will now allow for thorough studies on the influence of hypoxia-induced transcription on cell injury.

Having tight control over the “on” and “off” state of regulated gene expression systems is of great interest, and efforts to identify tet transactivator mutants with tighter drug-like behaviors are ongoing (Lamartina et al., “Stringent Control of Gene Expression In vivo by Using Novel Doxycycline-Dependent Trans-Activators,” Hum. Gene Ther. 13:199-210 (2002), which is hereby incorporated by reference in its entirety). One potential benefit of this approach would be the identification of rtTA variants with ligand binding constants several orders of magnitude lower than the affinity for ligand toward components of the apoptosome. However, as has been demonstrated, further optimization of these drug-responsive systems can also be achieved through the characterization of novel small-molecule ligands. As reported, 9-TB was more potent than doxycycline with regard to its ability to activate rtTA-driven gene expression from a stable reporter cell line. Presumably, this effect was related to its increased hydrophobicity and affinity for the tet transactivators (Zhu et al., “Silencing and Un-Silencing of Tetracycline-Controlled Genes in Neurons,” PLoS One 2:e533 (2007), which is hereby incorporated by reference in its entirety). Regarding the robust yet delayed kinetics of transgene expression observed with 9-TB, it was this property of the drug that allowed for overcoming ligand-mediated inhibition of hypoxia-induced apoptotic signaling seen with continuous dosing. Of note, although pulse dosing with doxycycline did ameliorate its suppressive effects on caspase cleavage, it did not achieve acceptable transgene expression levels using doxycycline after comparable brief pulse dosing. Based on these results, other tetracycline derivative compounds can be examined for their potential activity in the pulse-chase protocol.

The potential to capitalize on the “off-target” effects of dox and related compounds is recognized with regard to human disease. 9-TB has been used successfully in vivo to study the dynamic behavior of tet-responsive transgenic sequences in the central nervous system (Zhu et al., “Silencing and Un-Silencing of Tetracycline-Controlled Genes in Neurons,” PLoS One 2:e533 (2007), which is hereby incorporated by reference in its entirety). Given recent advances in the fields of cell- and viral-based therapeutics for neurodegenerative diseases and stroke, it is not far-fetched to consider using tetracycline-based gene regulation cassettes as drug delivery devices. In this context, having access to a drug capable of stimulating therapeutic gene expression while at the same time inhibiting endogenous apoptotic signaling programs represents a novel opportunity for therapeutic synergy. However, at the present time, tet-regulated gene expression systems are commonly used for commercial applications, including the large-scale production of therapeutically useful biologicals and in drug screening programs. Observations of the present invention have important implications in the latter setting where tightly regulated stable cell line systems are required for complex drug screening campaigns to identify pro- and anti-apoptotic compounds for cancer and neurodegenerative conditions. It is believed that the protocol modifications described herein will be easily translated to a wide variety of cell-based assays.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1. A method of using an inducible transgene in a cell, the method comprising:
providing a cell comprising an inducible transgene that expresses a gene product having activity on a cell death or cell survival pathway; and
exposing the cell to an agent that is capable of inducing the inducible transgene but also has direct activity on a cell death or cell survival pathway, said exposing being carried out in a manner effective to induce expression of the transgene while substantially minimizing said direct activity of the agent.

2. The method according to claim 1, wherein said exposing is carried out according to a pulse-washout schedule.

3. The method according to claim 2, wherein the pulse comprises carrying out said exposing for up to 12 hours.

4. The method according to claim 2 or 3, wherein the washout period comprises ceasing said exposing for at least 24 hours.

5. The method according to one of claims 2 to 4, wherein the pulse-washout schedule is repeated.

6. The method according to claim 1, wherein the inducible transgene comprises a tetracycline-responsive element (TRE) and the cell further comprises a fusion protein comprising a first polypeptide that binds to the TRE in the presence or absence of a substituted tetracycline compound and is operatively linked to a second polypeptide that regulates transcription in cells.

7. The method according to claim 6, wherein the agent is a substituted tetracycline compound.

8. The method according to claim 7, wherein the substituted tetracycline compound is doxycycline, 9-tert-butyl-doxycycline, chlortetracycline, oxytetracylcine, demeclocycline, methacycline, sancycline, minocycline, or tigecycline.

9. The method according to claim 1, wherein the gene product activates a cell death pathway.

10. The method according to claim 1, wherein the gene product activates a cell survival pathway.

11. The method according to claim 1, wherein the gene product and the agent have activity on the same cell death or cell survival pathway.

12. The method according to claim 1, wherein the gene product and the agent have activity on different cell death or survival pathways.

13. The method according to claim 1, wherein the gene product is a nucleic acid molecule.

14. The method according to claim 1, wherein the gene product is a polypeptide.

15. The method according to claim 1, wherein the cell is in vitro.

16. The method according to claim 15, wherein the cell is a cancer cell, a neuronal cell, a myocyte, or a somatic cell that exhibits selective vulnerability to either pharmacological or physiological toxins.

17. A kit comprising:
a response plasmid comprising (i) a promoter-effective nucleic acid molecule that includes a tetracycline-responsive element (TRE) and (ii) an insertion site suitable for introduction of an open reading frame of interest;
a regulator plasmid comprising a transgene that encodes a fusion protein comprising a first polypeptide that binds to the TRE in the presence or absence of a substituted tetracycline compound and is operatively linked to a second polypeptide that regulates transcription in cells; and
instructions for preparing a transgenic cell that can express the open reading frame of interest and the fusion protein, and exposing the transgenic cell to a substituted tetracycline compound according to a schedule that is effective to induce expression of the open reading frame of interest while substantially minimizing direct activity of the substituted tetracycline compound on a cell death or cell survival pathway.

18. The kit according to claim 17, further comprising the substituted tetracycline compound.

19. The kit according to claim 18, wherein the substituted tetracycline compound is doxycycline, 9-tert-butyl-doxycycline, chlortetracycline, oxytetracylcine, demeclocycline, methacycline, sancycline, minocycline, or tigecycline.

20. The kit according to claim 17, further comprising a cell line.

21-29. (canceled)
